(19) United States
(12) Patent Application Publication
(10) Pub. No.: US 2016/0355751 A1
(43) Pub. Date: Dec. 8, 2016

(54) DERIVATIVES OF 2-ETHYLIDENE NORBORNENE AND THEIR USE IN FRAGRANCE AND FLAVOR APPLICATIONS

(71) Applicant: TAKASAGO INTERNATIONAL CORPORATION (USA), Rockleigh, NJ (US)

(72) Inventors: Maureen BLANDINO, North Bergen, NJ (US); Michael E. LANKIN, High Bridge, NJ (US)

(21) Appl. No.: 15/170,933

(22) Filed: Jun. 1, 2016

Related U.S. Application Data
(60) Provisional application No. 62/168,998, filed on Jun. 1, 2015.

(51) Int. Cl. C11B 9/00; A61Q 19/00; A61K 8/92; A61K 8/33; A61K 8/34; A61K 8/37; A61K 8/35; A61K 8/40; A61K 8/31; A61Q 5/06; A61Q 19/10; A61Q 5/02; C11D 3/00; C11D 3/50; A23L 27/20; A61Q 13/00

(52) U.S. Cl. CPC C11B 9/0046; A61Q 13/00; A61Q 19/00; A61K 8/922; A61K 8/33; A61K 8/34; A61K 8/37; A61K 8/35; A61K 8/40; A61K 8/31; A61Q 5/065; A61Q 19/10; A61Q 5/02; C11D 3/001; C11D 3/50; A23L 27/204; A61K 2800/10; A61K 2800/592; A23V 2002/00

(57) ABSTRACT

Derivatives of 2-ethylidene norbornene are presented. The derivatives can be incorporated into fragrance or flavor compounds and consumer products. A method to impart, enhance or modify the odor properties of a perfuming composition or perfumed consumer product is also provided.

### CROSS-REFERENCE TO RELATED APPLICATIONS

This application claims priority to U.S. Provisional Patent Application Ser. No. 62/168,998, filed Jun. 1, 2015, the contents of which are incorporated by reference herein in its entirety.

### FIELD

The presently disclosed subject matter relates to derivatives of 2-ethylidene norbornene and their use in fragrance and flavor applications.

### BACKGROUND

There is a continuing interest in the preparation of new synthetic fragrance and flavor compounds with unique organoleptic properties and their use in consumer products. One strategy to prepare such compounds is to make derivatives of an easily-sourced chemical entity. 2-Ethylidene norbornene (ENB) is such a chemical entity.

ENB is a chemical entity commonly prepared via a Diels-Alder reaction with 1,3-cyclopentadiene and 1,3-butadiene, followed by isomerization of the external double bond. See, for example, Russian Journal of Organic Chemistry, 1998, 34 (10): 1403-1409; see also U.S. Patent Publication No. 2004/0049090, the disclosures of which are incorporated herein by reference in their entireties. ENB possesses two points of unsaturation, allowing for a variety of chemical reactions from which to prepare new families of materials. Hydroformylation of ENB is an example of one such chemical reaction. See, for example, European Patent 0108504; U.S. Pat. No. 4,524,017; and Japanese patent publication nos. JP6242904, and JP59110643, the disclosures of which are incorporated herein by reference in their entireties. It is known in the art that the endocyclic alkene of ENB is more reactive to the hydroformylation process than the exocyclic alkene. Further, the ring alkene of ENB can be selectively hydroformylated with a catalyst in the presence of a ligand at specific pressures and temperatures, while keeping the exocyclic alkene intact.

Synthesis of unique compounds with the ability to impart organoleptic properties on fragrance and flavor compounds, such as specific ENB derivatives, is not known in the art. There is also a need in the art to provide cheaper alternative compounds that provide similar organoleptic properties to their known counterparts. For example, perilla aldehyde (CAS 2111-75-3) is commonly used as a fragrance and flavor agent. However, the amount of perilla aldehyde that can be incorporated into a product is strictly limited to 0.1% in a finished product due to side effects such as skin irritation. Further, the agent is an expensive additive for commercial products, often exceeding $700 per kilogram. A cheaper, safer, compound with a similar organoleptic profile is therefore desirable. The presently disclosed subject matter addresses this deficiency.

### SUMMARY

The presently disclosed subject matter provides a mixture of compounds of Formula I and Formula II:

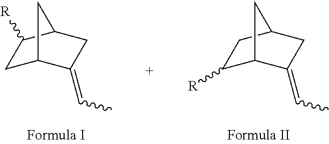

wherein R is selected from the group consisting of CH(CH₃)(OH), CH(CH₂CH₃)(OH), CH(CHCH₂)(OH), CH(CH(CH₃)₂)(OH), CH(CH₃)(OC(O)CH₃), CH(CH₂CH₃)(OC(O)CH₃), CH(CHCH₂)(OC(O)CH₃), CH(CH(CH₃)₂)(OC(O)CH₃), C(O)CH₃, C(O)CH₂CH₃, C(O)CHCH₂, C(O)(CH(CH₃)₂), CHNOH, CHC((CH₃)(CHO)), CHC((CH₃)(CH₂OH)), CHCHCH₂CHCHCH₂CH, CH₂OCH₂CHCH₂, CH₂OCH₂C((CH₂)(CH₃)), CH₂OCH₂CH₂CH₂CHO, CH₂OCH₂CH₂CH₂CH₂OH, CH₂OCH((CH₃)(OCH₂CH₃)), CH₂OCOCH(CH₃)₂, CH₂OCOOCH₃, and CH₂OCOOCH₂CH₃, and stereoisomers thereof.

In certain embodiments, R is CH(CH₃)(OH), CH(CH₂CH₃)(OH), CH(CH₃)(OC(O)CH₃), or CH₂OCH₂CH₂CH₂CHO.

The presently disclosed subject matter also provides fragrance composition comprising:

a) a mixture of compounds of Formula I and Formula II:

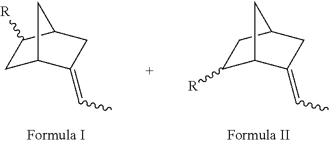

wherein R is selected from the group consisting of CH(CH₃)(OH), CH(CH₂CH₃)(OH), CH(CHCH₂)(OH), CH(CH(CH₃)₂)(OH), CH(CH₃)(OC(O)CH₃), CH(CH₂CH₃)(OC(O)CH₃), CH(CHCH₂)(OC(O)CH₃), CH(CH(CH₃)₂)(OC(O)CH₃), CN, C(O)CH₃, C(O)CH₂CH₃, C(O)CHCH₂, C(O)(CH(CH₃)₂), CHNOH, CHC((CH₃)(CHO)), CHC((CH₃)(CH₂OH)), CHCHCH₂CHCHCH₂CH, CH₂OCH₂CHCH₂, CH₂OCH₂C((CH₂)(CH₃)), CH₂OCH₂CH₂CH₂CHO, CH₂OCH₂CH₂CH₂CH₂OH, CH₂OCH((CH₃)(OCH₂CH₃)), CH₂OCOCH(CH₃)₂. CH₂OCOOCH₃, and CH₂OCOOCH₂CH₃, and stereoisomers thereof; and b) at least one perfume ingredient selected from the group consisting of one or more bases, solvents and combinations thereof.

In certain embodiments, the mixture of compounds of Formula I and Formula II are present in the fragrance composition at an amount of from about 0.01% to about 50% w/w.

In certain embodiments, the mixture of compounds of Formula I and Formula II are present in the fragrance composition at an amount of from about 0.1% to about 10% w/w.

In further embodiments, the mixture of compounds of Formula I and Formula II are present in the fragrance composition at an amount of about 5% w/w.

In other embodiments, the mixture of compounds of Formula I and Formula II are present in the fragrance composition at an amount of about 2% w/w.

In certain embodiments, R is CN.

In further embodiments, the at least one perfume ingredient is solvent. In certain embodiments, the solvent is a natural solvent. In certain embodiments, the natural solvent is selected from the group consisting of vegetable oils, sunflower oil, almond oil, olive oil, avocado oil, soybean oil, safflower oil, mineral oil, orange oil, and combinations thereof.

The presently disclosed subject matter also provides a consumer product comprising the fragrance composition.

In certain embodiments, the product is selected from the group consisting of cologne, perfume, body sprays, lotions, creams, body washes, hand soaps, shampoos, conditioners, soaps, deodorants, antiperspirants, cosmetics, shampoo, conditioner, and bath agents.

The presently disclosed subject matter also provides a method to enhance or modify the organoleptic properties of a consumer product, which method can include contacting the consumer product with a fragrance composition.

The presently disclosed subject matter also provides a flavor composition comprising:

a) a mixture of compounds of Formula I and Formula II:

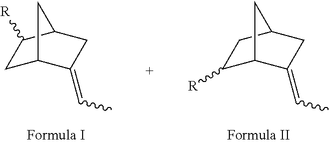

wherein R is selected from the group consisting of CH(CH₃)(OH), CH(CH₂CH₃)(OH), CH(CHCH₂)(OH), CH(CH(CH₃)₂)(OH), CH(CH₃)(OC(O)CH₃), CH(CH₂CH₃)(OC(O)CH₃), CH(CHCH₂)(OC(O)CH₃), CH(CH(CH₃)₂)(OC(O)CH₃), CN, C(O)CH₃, C(O)CH₂CH₃, C(O)CHCH₂, C(O)(CH(CH₃)₂), CHNOH, CHC((CH₃)((CHO)), CHC((CH₃)(CH₂OH)), CHCHCH₂CHCHCH₂CH, CH₂OCH₂CHCH₂, CH₂OCH₂C((CH₂)(CH₃)), CH₂OCH₂CH₂CH₂CHO, CH₂OCH₂CH₂CH₂CH₂OH, CH₂OCH((CH₃)(OCH₂CH₃)), CH₂OCOCH(CH₃)₂, CH₂OCOOCH₃, and CH₂OCOOCH₂CH₃, and stereoisomers thereof; and b) one or more flavor co-ingredients.

The presently disclosed subject matter further provides methods to impart, enhance or modify the odor properties of a perfuming composition or perfumed consumer product, which method comprises adding to said composition or product a mixture of compounds of Formula I and Formula II:

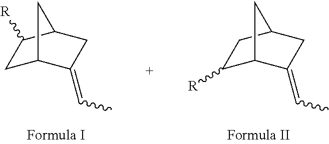

wherein R is selected from the group consisting of CH(CH₃)(OH), CH(CH₂CH₃)(OH), CH(CHCH₂)(OH), CH(CH(CH₃)₂)(OH), CH(CH₃)(OC(O)CH₃), CH(CH₂CH₃)(OC(O)CH₃), CH(CHCH₂)(OC(O)CH₃), CH(CH(CH₃)₂)(OC(O)CH₃), CN, C(O)CH₃, C(O)CH₂CH₃, C(O)CHCH₂, C(O)(CH(CH₃)₂), CHNOH, CHC((CH₃)(CHO)), CHC((CH₃)(CH₂OH)), CHCHCH₂CHCHCH₂CH, CH₂OCH₂CHCH₂, CH₂OCH₂C((CH₂)(CH₃)), CH₂OCH₂CH₂CH₂CHO, CH₂OCH₂CH₂CH₂CH₂OH, CH₂OCH((CH₃)(OCH₂CH₃)), CH₂OCOCH(CH₃)₂, CH₂OCOOCH₃, and CH₂OCOOCH₂CH₃, and stereoisomers thereof; and wherein the mixture of compounds imparts odor notes of a perilla type.

In certain embodiments, the mixture of compounds of Formula I and Formula II are present in the composition or product at an amount of from about 0.01% to about 50% w/w.

In certain embodiments, the mixture of compounds of Formula I and Formula II are present in the composition or product at an amount of from about 0.1% to about 10% w/w.

In further embodiments, the mixture of compounds of Formula I and Formula II are present in the composition or product at an amount of about 5% w/w.

In other embodiments, the mixture of compounds of Formula I and Formula II are present in the composition or product at an amount of about 2% w/w.

In certain embodiments, R is CN.

The foregoing has outlined rather broadly the features and technical advantages of the presently disclosed subject matter in order that the detailed description that follows may be better understood. Additional features and advantages of the subject matter will be described hereinafter which form the disclosed subject matter. It should be appreciated by those skilled in the art that the conception and specific embodiments disclosed may be readily utilized as a basis for modifying or designing other structures for carrying out the same purposes of the presently disclosed subject matter. It should also be realized by those skilled in the art that such equivalent constructions do not depart from the spirit and scope of the subject matter as set forth in the appended claims. The novel features which are believed to be characteristic of the subject matter, both as to its organization and method of operation, together with further objects and advantages will be better understood from the following description.

### DETAILED DESCRIPTION

As noted above, there remains a need in the art for new synthetic fragrance and flavor compounds with unique organoleptic properties. The presently disclosed subject matter addresses this need through the preparation of new compounds derived from ENB and preparation of fragrance and/or flavor compositions comprising said compounds.

### 1. Definitions

As used herein, the words “a” or “an,” when used in conjunction with the term “comprising” in the claims and/or the specification, may mean “one,” but they are also consistent with the meaning of “one or more,” “at least one,” and/or “one or more than one.” Furthermore, the terms “having,” “including,” “containing” and “comprising” are interchangeable, and one of skill in the art will recognize that these terms are open ended terms.

As used herein, the term “about” or “approximately” means within an acceptable error range for the particular value as determined by one of ordinary skill in the art, which will depend in part on how the value is measured or determined, i.e., the limitations of the measurement system. For example, “about” can mean a range of up to 20%, up to 10%, up to 5%, and or up to 1% of a given value.

As used herein, the terms “consumer product” or “end product” refers to a composition that is in a form ready for use by the consumer for the marketed indication. A suitable solvent for use in a consumer product is a solvent that when combined with other components of the end product, will not render the consumer product unfit for its intended consumer use.

As used herein, the terms “flavor composition” refers to a composition that contains one or more compound(s) (e.g., co-ingredients) that provide(s) a desired taste when combined with a solvent that is suitable for oral administration and oral consumption. One of ordinary skill in the art of flavors is able to select them on the basis of its general knowledge and according to the nature of the product to be flavored and the desired taste.

As used herein, the term “fragrance” can also be used interchangeably with scent or odor.

As used herein, the terms “fragrance composition” refers to a mixture comprising one or more fragrance components, in any of their forms, and one or more solvents or perfuming co-ingredients. As known in the art, a fragrance composition will contain one or more fragrance components (e.g., perfuming co-ingredients) in order to impart an olfactory note to the composition (e.g., a household cleaner, perfume, or other commercial product) to which it is added.

In general terms, flavor and perfuming co-ingredients belong to chemical classes as varied as alcohols, aldehydes, ketones, esters, ethers, acetates, nitriles, terpene hydrocarbons, nitrogenous or sulphurous heterocyclic compounds and essential oils of natural or synthetic origin, and are well known to those of ordinary skill in the art. Many of these ingredients are listed in reference texts such as S. Arctander, Perfume and Flavor Chemicals, 1969, Montclair, N.J., USA (or any of its more recent versions), which is herein incorporated by reference in its entirety.

As used herein, the term “enantiomers” refers to a pair of stereoisomers that are non-superimposable mirror images of each other. A 1:1 mixture of a pair of enantiomers is a “racemic” mixture. The term is used to designate a racemic mixture where appropriate.

As used herein, the term “diastereoisomers” refers to stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro or levorotatory) which they rotate plane polarized light at the wavelength of the sodium D line. The compounds of the presently disclosed subject matter contain one or more asymmetric centers and may thus give rise to enantiomers, diastereomers, and other stereoisomeric forms that may be defined, in terms of absolute stereochemistry, as (R)- or (S)-. The presently disclosed subject matter is meant to include all such possible isomers, including racemic mixtures, optically pure forms and intermediate mixtures. Optically active (R)- and (S)-isomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

As used herein, the term “isomers” refers to different compounds that have the same molecular formula but differ in arrangement and configuration of the atoms. Also as used herein, the term “stereoisomer” refers to any of the various stereo isomeric configurations which may exist for a given compound of the presently disclosed subject matter and includes geometric isomers. It is understood that a substituent may be attached at a chiral center of a carbon atom. Therefore, the presently disclosed subject matter includes enantiomers, diastereomers or racemates of the compound. Also as used herein, the terms “constitutional isomers” refers to different compounds which have the same numbers of, and types of, atoms but the atoms are connected differently.

### 2. 2-ethylidene norbornene compounds

The presently disclosed subject matter includes a series of novel compounds derived from ENB, shown below.

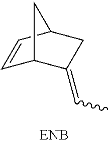

As used herein, “ENB” refers to 2-ethylidene norbornene, or 5-ethylidene-2-norbornene, or (1S,4S)-5-ethylidenebicyclo[2.2.1]hept-2-ene.

ENB is a chemical entity commonly prepared via a Diels-Alder reaction with 1,3-cyclopentadiene and 1,3-butadiene, followed by isomerization of the external double bond. It is commercially available from several sources including, but not limited to, Acros Organics, Alfa Aesar, Sigma-Aldrich, and TCI America. ENB possesses two points of unsaturation, allowing for a variety of chemical reactions from which to prepare new families of materials. In certain embodiments, these materials are of particular interest for use in fragrances and/or flavors.

In certain embodiments, the endocyclic alkene of ENB is more reactive to the hydroformylation process than the exocyclic alkene. In certain embodiments, the ring alkene of ENB is selectively hydroformylated with a catalyst in the presence of a ligand at specific pressures and temperatures, while keeping the exocyclic alkene intact. For example, when ENB undergoes the hydroformylation, a mixture of the compounds of Formula I and Formula II, wherein R is CHO is synthesized as follows:

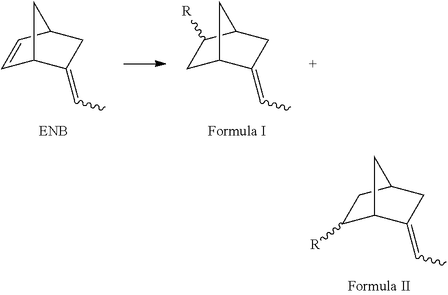

In certain non-limiting embodiments, the catalyst can be, but is not limited to: HRh(CO)PPh₃, Rh(CO)₂Acac, [Rh(COD)Cl]₂, Rh(COD)Acac, RhCl(PPh₃)₃, Rh(COD)BF₄(bis-diethylphosphalanobenzene), RhCl₃, Co₂(CO), and Fe(CO)s. In certain embodiments, the amount of catalyst can be from about 0.00001 molar equivalents to about 0.001 molar equivalents, or from about 0.0001 molar equivalents to about 0.001 molar equivalents. In specific embodiments, the amount of catalyst is about 0.0002 molar equivalents.

In further embodiments, the ligand can be, but is not limited to PPh₃, P(OPh)₃, P(OMe)₃, P(3-MeOPh)₃, BiPhePhos, BINAP, SegPhos, DTBM-SegPhos, P(O-2,4-di-t-butylPh)₃, NIXANTPHOS and PBu₃. In certain embodiments, the amount of ligand can range from about 0.001 molar equivalents to about 0.1 molar equivalents, or from about 0.01 molar equivalents to about 0.1 molar equivalents. In some embodiments, the amount of ligand is about 0.015 molar equivalents.

In further embodiments, the pressure can be, but is not limited to from about 100 to about 350 psi, or from about 200 psi to about 350 psi. In some embodiments, the pressure is about 300 psi. This pressure is specific to avoid reaction of the exocyclic alkene.

In certain embodiments, reaction temperatures range from about 50° C. to about 110° C., or from about 70° C. to about 110° C., or from about 90° C. to about 110° C., or from about 100° C. to about 110° C. In specific embodiments, the reaction temperature is about 105° C.

In certain embodiments, the reaction can be performed neat. In other embodiments the reaction can be performed with solvents including, but not limited to, toluene, water, tetrahydrofuran, cyclohexane, dichloromethane, methanol, and mixtures thereof.

In certain embodiments the mixture of compounds of Formula I and Formula II is synthesized wherein R is: CH(CH₃)(OH), CH(CH₂CH₃)(OH), CH(CHCH₂)(OH), CH(CH(CH₃)₂)(OH), CH(CH₃)(OC(O)CH₃), CH(CH₂CH₃)(OC(O)CH₃), CH(CHCH₂)(OC(O)CH₃), CH(CH(CH₃)₂)(OC(O)CH₃), C(O)CH₃, C(O)CH₂CH₃, C(O)CHCH₂, C(O)(CH(CH₃)₂), CHNOH, CN, CHC((CH₃)(CHO)), CHC((CH₃)(CH₂OH)), CHCHCH₂CHCHCH₂CH, CH₂OCH₂CHCH₂, CH₂OCH₂C((CH₂)(CH₃)), CH₂OCH₂CH₂CH₂CHO, CH₂OCH₂CH₂CH₂CH₂OH, CH₂OCH((CH₃)(OCH₂CH₃)), CH₂OCOCH(CH₃)₂, CH₂OCOOCH₃, and CH₂OCOOCH₂CH₃, including constitutional isomers, enantiomers, stereoisomers and racemic mixtures of said compounds.

In some embodiments, R is CHO and the mixture is further reacted with Grignard reagents. Grignard reagents can comprise the formula MgR₁X, where R₁=methyl, ethyl, vinyl, or isopropyl. In certain embodiments, X═Cl or Br. In certain embodiments, reacting the mixture of compounds with a Grignard reagent forms new compounds of the invention of the formulas (I) and (II) where R═CH(CH₃)(OH), CH(CH₂CH₃)(OH), CH(CHCH₂)(OH) and CH(CH(CH₃)₂)(OH), including constitutional isomers, enantiomers, stereoisomers and racemic mixtures of said compounds.

Synthesized compounds and the organoleptic properties thereof are described in Table 1.

TABLE 1

| | | Organoleptic |
|---|---|---|
| R | Structure | Description |
| CH(CH₃)(OH) | 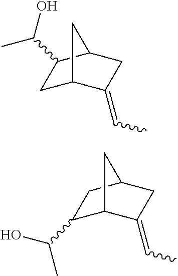 | Floral, slightly muguet, fresh, lilac, herbaceous, minty |
| CH(CH₂CH₃)(OH) | 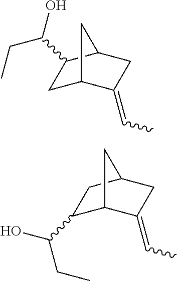 | Green, fruity, magnol, woody, violet leaf |
| CH(CHCH₂)(OH) | 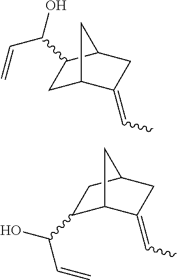 | Root vegetable, mushroom, potato |
| CH(CH(CH₃)₂)(OH) | 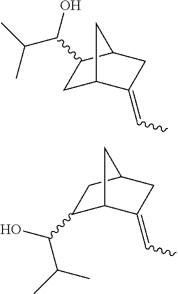 | Weak, rubbery |

The compounds listed in Table 1 are alcohols. In certain embodiments, these new compounds can be acetylated by means commonly known in the art. In one non-limiting example, the compounds can be refluxed with acetic anhydride in toluene to synthesize four new entities of the invention of formulas (I) and (II) where R═CH(CH₃)(OC(O)CH₃), CH(CH₂CH₃)(OC(O)CH₃), CH(CHCH₂)(OC(O)CH₃) and CH(CH(CH₃)₂)(OC(O)CH₃), including constitutional isomers, enantiomers, stereoisomers and racemic mixtures of said compounds. The organoleptic properties of these new acetyl esters are described in Table 2.

TABLE 2

| | | Organoleptic |
|---|---|---|
| R | Structure | Description |
| CH(CH₃)(OC(O)CH₃) | 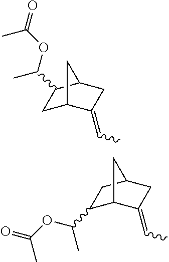 | Grapefruit, lemongrass, vetivert acetate |
| CH(CH₂CH₃)(OC(O)CH₃) | 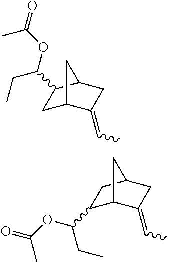 | Woody, grapefruit, moss, vetivert acetate |
| CH(CHCH₂)(OC(O)CH₃) | 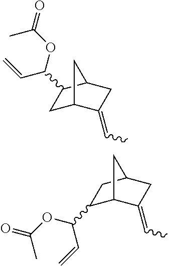 | Rhubarb, grapefruit, citrus, vetivert acetate |
| CH(CH(CH₃)₂)(OC(O)CH₃) | 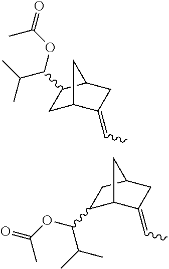 | Grapefruit |

In other embodiments, the compounds of Table 1 can be oxidized by usual means known in the art. In one non-limiting example, the compounds can be reacted with an oxidizing agent such as (bisacetoxy)iodobenzene (BAIB) with (2,2,6,6-Tetramethylpiperidin-1-yl)oxyl (TEMPO) catalyst in dichloromethane at room temperature, to prepare four new ketones of the invention of formulas (I) and (II) where R═C(O)CH₃, C(O)CH₂CH₃, C(O)CHCH₂ and C(O)(CH(CH₃)₂), including constitutional isomers, enantiomers, stereoisomers and racemic mixtures of said compounds. The organoleptic properties of these new ketones are described in Table 3.

TABLE 3

| | | Organoleptic |
|---|---|---|
| R | Structure | Description |
| C(O)CH₃ | 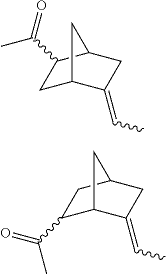 | Sulfury, spicy, fruity, strong |
| C(O)CH₂CH₃ | 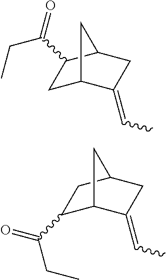 | Yuzu-like, grapefruit, mandarin |
| C(O)CHCH₂ | 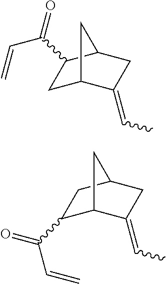 | Fruity, weak |
| C(O)(CH(CH₃)₂) | 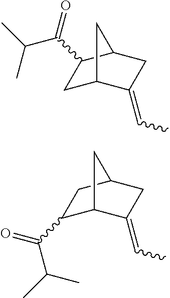 | Green, bitter, fruity |

In further embodiments, the compounds of formulas (I) and (II) where R═CHO can be subjected to other known chemical reactions, as described in the Examples below, to give compounds of the invention of the formulas (I) and (II) where R═CHNOH, CN, CHC((CH₃)(CHO)), CHC((CH₃)(CH₂OH)) and CHCHCH₂CHCHCH₂CH, including constitutional isomers, enantiomers, stereoisomers and racemic mixtures of said compounds. Organoleptic properties of these compounds are described in Table 4.

TABLE 4

| | | Organoleptic |
|---|---|---|
| R | Structure | Description |
| CHNOH | 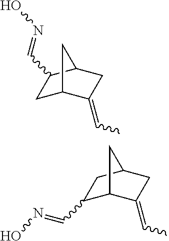 | Paper-like |
| CN | 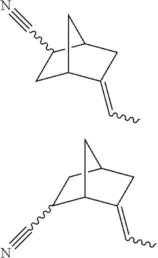 | Gourmand, spicy, cinnamon, cherry, almond, shiso, herbaceous, perilla aldehyde, cuminic |
| CHC((CH₃)(CHO)) | 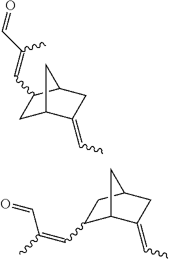 | Citrus, grapefruit, unripe apricot, green, sour |
| CHC((CH₃)(CH₂OH)) | 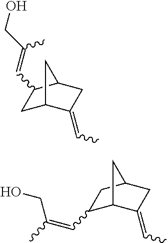 | Woody |
| CHCHCH₂CHCHCH₂CH | 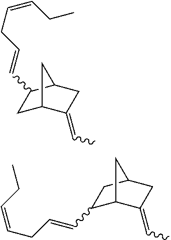 | Honey |

In other embodiments, treating the compounds of formulas (I) and (II) where R═CHO with a reducing agent, such as sodium borohydride, can synthesize compounds of formulas (I) and (II) where R═CH₂OH, including enantiomers, steriosomers and racemic mixtures of said compounds. This material, a mixture of alcohols ((1 S,4S)-5-ethylidenebicyclo[2.2.1]heptan-2-yl)methanol and ((1S,4R)-6-ethylidenebicyclo[2.2.1]heptan-2-yl)methanol, can be further reacted to form lower ethers and esters (methyl, ethyl and propyl ethers, and formyl, acetyl and propionyl esters), including constitutional isomers, enantiomers, stereoisomers and racemic mixtures thereof, as described in Table 5.

TABLE 5

| | | Organoleptic |
|---|---|---|
| R | Structure | Description |
| CH₂OCH₂CHCH₂ | 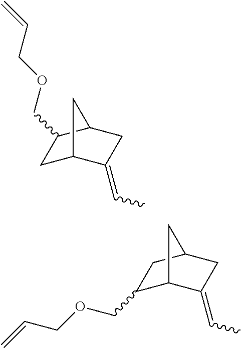 | Green, fruity, oily, canned pineapple, metallic |
| CH₂OCH₂C((CH₂)(CH₃)) | 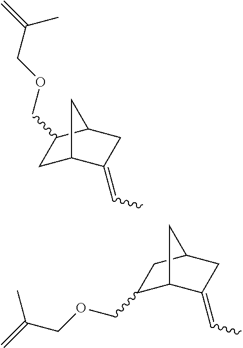 | Bitter, licorice |
| CH₂OCH₂CH₂CH₂CHO | 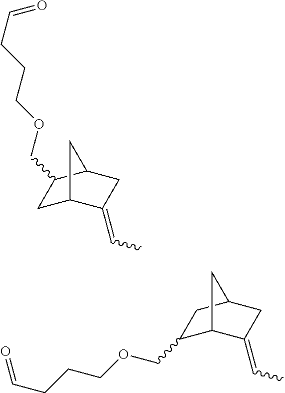 | Green, muguet, floral, sweet, heavy |
| CH₂OCH₂CH₂CH₂CH₂OH | 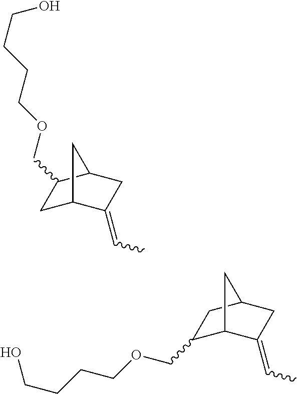 | Weak, floral |
| CH₂OCH((CH₃)(OCH₂CH₃)) | 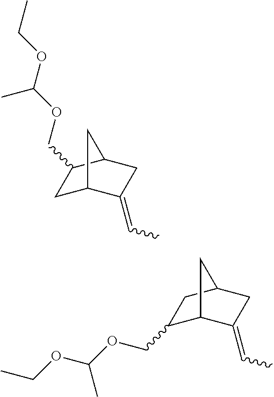 | Hyacinth, green, soapy, licorice |
| CH₂OCOCH(CH₃)₂ | 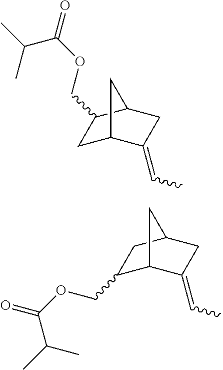 | Fatty, waxy, fruity |
| CH₂OCOOCH₃ | 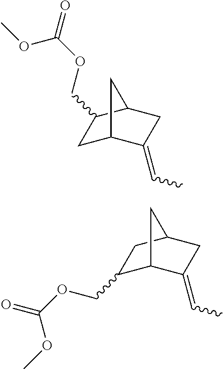 | Fruity, berry |
| CH₂OCOOCH₂CH₃ | 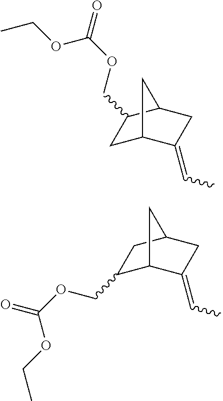 | Fruity, berry |

In certain embodiments, the mixture of synthesized compounds of Formula I and Formula II can be incorporated into fragrance and/or flavor compositions.

### 3. Fragrance Compositions, Combinations, and Products

The presently disclosed subject matter provides fragrance compositions that can include one or more mixtures of compounds of Formula I and Formula II.

Fragrance compositions can contain one, two, three, four or more mixtures of compounds of Formula I and Formula II. In certain embodiments, a fragrance composition comprises two mixtures of compounds of Formula I and Formula II. In certain embodiments, a fragrance composition comprises three mixtures of compounds of Formula I and Formula II.

As embodied in the non-limiting Examples, mixtures of compounds of Formula I and Formula II can be incorporated into fragrance compositions in various amounts. By way of non-limiting example, one or more mixtures of compounds of Formula I and Formula II can be incorporated into a fragrance composition at an amount of from about 0.01% to about 50%, from about 0.1% to about 40%, from about 1% to about 30%, from about 2% to about 25%, or from about 5% to about 15% w/w. In other embodiments, one or more mixtures of compounds of Formula I and Formula II can be incorporated into a fragrance composition at an amount of from about 0.01% to about 10% or from about 0.1% to about 10% w/w, or from about 1.0% to about 10% w/w, or from about 3% to about 8%. In certain embodiments, one or more mixtures of compounds of Formula I and Formula II can be incorporated into a fragrance composition at an amount of about 5% w/w.

In certain embodiments, the fragrance composition can comprise a mixture of compounds of Formula I and Formula II as well as one or more bases, solvents and combinations thereof.

In certain embodiments, bases can include, but are not limited to, essential oils, lactones, aldehydes, alcohols, ketones, nitriles, esters, amides, oximes, and other fragrant compounds and perfuming co-ingredients.

In certain embodiments, the solvents can include, but are not limited to, diproplyene glycol, propylene glycol, dieth-phthalate (DEP), diisononyl phthalate (DINP), benzyl benzoate, benzyl alcohol, iso propyl myristate (IPM), isopropyl palmitate (IPP/Deltyl Prime), butyl stearate, dioctyl adipate, triethyl citrate, methyl hydrogenated rosinate (CAS No. 8050-15-5),

terpenes (e.g., Glidsol 100), paraffinic naphthenic solvent (e.g., LPA-170 Solvent), isoalkanes (e.g., Soltrol 170 Isoparaffin), isoparaffins, isooctadecanol, (e.g., Tego Alkanol 66), phenoxyethanol, diethylene glycol monoethyl ether (Carbitol low gravity), glycol ether (Methyl Carbitol), Dipropylene Glycol Methyl Ether (e.g., Dowanol DPM), Dipropylene Glycol Methyl Ether Acetate (e.g., Dowanol DPMA), Propylene glycol methyl ether (e.g., Dowanol PM Glycol Ether), Tripropylene Glycol Methyl Ether, Diisoheptyl Phthalate (e.g., Jayflex® 77 available from Exxon), deionized or distilled water, specially denatured ethyl alcohol (e.g., SDA 40B), Dimethyl Adipate/Dimethyl Glutarate (e.g., DBE®-LVP Esters available from FLEXISOLV®), Racemic mixture (+/−)-2,2-dimethyl-4-hydroxymethyl-1,3-dioxolane (e.g., Augo Clean Multi Solvent), Alcohol 40B Anhydrous 200 Proof, alcohol SDA 40B 190 Proof, Triacetin, 3-Methoxy-3-methyl-1-butanol (Solfit), Benzyl Laurate, Tripropylene Glycol Methyl Ether (e.g., Dowanol TPM), Dipropylene glycol n-butyl ether (e.g., Dowanol DPNB), Dimethyl siloxane, trimethylsiloxy-terminated (e.g., Dowanol Corning 200 Fluid), Caprylic/Capric Triglycerides (e.g., Neobee M-5), propylene glycol and glyceryl oleate (e.g., Arlacel 186), Uniceth-IC20L (e.g., Arlasolve 200 L), propanediol, 1, 3, Butyl Cellosolve, Hexylene glycol, Glycerine, N Methyl Stearate, Isopropyl alcohol, 2-Methyl-1,3-propanediol (e.g., MP Diol Glycol), Diethyl Citrate, Triethyl Acetyl Citrate, Isopentyldiacetate (IPD-AC, Dimethyl 2-methylpentanedioate
(e.g., Rhodiasolv Iris), medium chain triglicyrides (MTC), terpene hydrocarbons (e.g., Dipentene 5100, DL-limonene (e.g., Dipentene 122), 3,5,5-trimethylhexyl acetate, Diethyl Malonate, Limonene (e.g., Unitene D), cyclohexyl acetate, para-tertiary-butyl (e.g., Vertenex), Ethyl Acetate, Diethyl Succinate.

In certain embodiments, the solvent can be natural solvents including, but not limited to, vegetable oils, including sunflower oil, almond oil, olive oil, avocado oil, soybean oil, safflower oil, mineral oil, orange oil.

In certain embodiments, the presently disclosed fragrance compositions can present one or more notes selected from the group consisting of floral, muguet (lily of the valley), fresh, lilac, herbaceous, minty, green, woody, violet leaf, root vegetable, mushroom, potato, weak, rubbery, grapefruit, lemongrass, vetivert acetate, moss, rhubarb, citrus, sulfury, spicy, strong, yuzu-like, mandarin, bitter, paper-like, gourmand, cinnamon, cherry, almond, shiso or perilla, perilla aldehyde, cuminic or cumin-like, unripe apricot, sour, honey, oily, canned pineapple, metallic, licorice, hyacinth, soapy, waxy, fruity, or berry. In certain embodiments, the presently disclosed fragrance compositions can have a shiso/perilla type note. In certain embodiments, the presently disclosed fragrance compositions can have a berry rhubarb type note.

In certain embodiments, the mixture of compounds of Formula I and Formula II can be used to reconstruct the scent profiles of natural citrus oils used in perfume compositions. For example, naturally occurring citrus oils such as bergamot and orange contain perilla aldehyde. In certain embodiments, the presently disclosed fragrance compositions can be constructed without perilla aldehyde and with a mixture of compounds of Formula I and Formula II. In certain embodiments, the fragrance composition comprises a mixture of (1S,4S)-5-ethylidenebicyclo[2.2.1]heptane-2-carbonitrile and (1S,4R)-6-ethylidenebicyclo[2.2.1]heptane-2-carbonitrile.

In certain embodiments, the presently disclosed fragrance compositions and combinations can be incorporated into fragrance products and applications. The compositions and combinations of the presently disclosed subject matter can be used to impart improved olfactory effects to consumers in various products, including but not limited to aerosol air freshening products, gel air freshening products, candles, fragranced wax melts, potpourri, piezo-electric fragrance devices, reed diffusers, fabrics, liquid electrical air fresheners, powered evaporative air fresheners, filter papers, laminated cardboard, membrane diffusers, ceramic diffusers, spray refresheners, and the like.

In certain embodiments, the presently disclosed fragrance compositions can be incorporated into consumer products including, but are not limited to, household or home care products. Non-limiting examples of such products include home cleaning products (e.g., hand and auto dish cleaners, hard surface cleaners, laundry products such as laundry detergents, softeners, cleaners, dryer sheets, etc.); pet care products (e.g., cat litter); sanitary products (e.g., towels, toilet paper, tissue paper, wet tissue paper, handkerchiefs, wet towels, etc.); writing products (e.g., pens, crayons, paints, pencils, paper, origami, seals, etc.); auto care products (e.g., cleaners, air fresheners, wipes, soaps, etc.); products for play (e.g., balls, beanbags, cards, tops, dolls, building blocks, etc.).

In certain embodiments, the presently disclosed fragrance compositions and combinations can be incorporated into bathroom cleaning products, including but not limited to, bath or tile cleaner, toilet cleaner, sink cleaner, disinfectants, antimicrobial products, mildew cleaners, etc.

In certain embodiments, products include those applied to or brought into contact with a consumer's skin and body heat. Products include fine fragrance (e.g., cologne, perfume, body sprays); personal care products (e.g., lotions, creams, body washes, hand soaps, shampoos, conditioners, soaps, deodorants, antiperspirants, etc.); cosmetics (e.g., skin cream, cleansing cream, night cream, hand cream, lotion, after-shave lotion, shaving creams, gels and foams, body lotion, foundation, lip stick, lip cream, nail polish, nail polish remover, talcum powder, anti-wrinkle and/or anti-aging cosmetics, sun protection products, sun-burn lotions, massage oil, etc.); hair cosmetics (e.g., shampoo, rinse, conditioner, rinse in shampoo; hair styling agents such as pomade, hair tonic, hair gel, hair cream and hair mousse; hair growing agents; hair coloring agents; etc.); and bath agents (e.g., powder bath additives, solid foaming bath additives, bath oils, bubble bath aroma generators, bath salts, etc.).

In certain embodiments, fine fragrance compositions known in the art can be reconstructed containing a mixture of compounds of Formula I and Formula II to produce cheaper and/or safer fragrance compositions. For example, a mixture of compounds of Formula I and Formula II as presently disclosed can be used as substitutes for the compounds used in fragrances described by U.S. Pat. No. 8,304,380 or U.S. Pat. No. 8,691,746, the disclosures of which are herein incorporated by reference in their entireties. For example, a mixture of compounds of Formula I and Formula II as presently disclosed can be used as substitutes for the known Shisolia™ captive (4-vinylcyclohex-1-enecarbaldehyde).

In certain embodiments, the presently disclosed fragrance compositions and combinations can be used to enhance or modify the organoleptic properties of a consumer product.

### 4. Flavor Compositions, Combinations, and Products

The presently disclosed subject matter provides flavor compositions that can include one or more mixtures of compounds of Formula I and Formula II, one or more flavor compounds, and/or one or more supports. Mixtures of compounds of Formula I and Formula II can be incorporated into flavor compositions in various amounts.

In certain embodiments, the presently disclosed flavor compositions and combinations can be incorporated into various consumer products, including flavored products. For example, the flavor compositions and combinations can be incorporated into products, together with co-ingredients or adjuvants, for example, to impart taste and/or improved gustatory effects to consumers. Such products include, but are not limited to: foodstuffs such as baked goods, dairy products, desserts, etc.; beverages such as juices, sodas, teas, flavored waters, fruit-based “smoothy” drinks, milk-based drinks, alcoholic beverages, energy drinks, drink powders, etc.; confectionaries such as sweets, hard candy, chewing gums; and condiments, spices, flavorings, seasonings, dry cereals, oatmeals, granola bars, gelatinous materials, and snacks.

In certain embodiments, the flavor compositions and combinations can be incorporated into oral products or pharmaceuticals. Oral products include tooth paste, tooth gel, oral wash, mouth rinse, mouthwash, oral film strips, etc. Pharmaceuticals include plasters, ointments, lotions, analgesics, liniments, decongestants, cough mixtures, throat lozenges, indigestion preparations, oral analgesics, tablets, capsules, drops, etc. In certain embodiments, a flavor composition or combination of the present invention can be added, directly or indirectly, to a pharmaceutical dosage form, such as a tablet, capsule, drop or lozenge, that also contains a therapeutically active agent.

In certain embodiments, the flavor composition can comprise a mixture of compounds of Formula I and Formula II as well as one or more bases, solvents and combinations thereof.

In certain embodiments, bases can include, but are not limited to, essential oils, lactones, aldehydes, alcohols, ketones, and other flavor compounds and flavor co-ingredients.

### EXAMPLES

The presently disclosed subject matter will be better understood by reference to the following Examples, which are provided as exemplary of the invention, and not by way of limitation.

In the following Examples, the temperatures are indicated in degrees centigrade (° C.); pressure is indicated in torr (T); the NMR spectral data were recorded in CDCl₃ with a 500 MHz machine for ¹H and ¹³C, and the chemical displacements are indicated in ppm with respect to TMS as the standard. All reactions were carried out under a blanket of N₂ gas.

### Example 1

This Example illustrates the synthesis of the mixture of (1S,4S)-5-ethylidenebicyclo[2.2.1]heptane-2-carbaldehyde and (1S,4R)-6-ethylidenebicyclo[2.2.1]heptane-2-carbaldehyde:

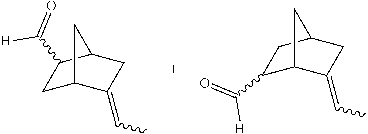

ENB (100 g), RhH(CO)(PPh₃)₃ (8.4 mg, 0.00001 molar equivalents) and triphenyl phosphine (2.4 g, 0.011 molar equivalents) were sealed in an autoclave. The vessel was purged with a mixture of 1:1 CO:H₂ gases, then filled to 300 psi and heated to 105° C. After 3.5 hours, the mono-hydroformylation was complete. The product (102.8 g) was isolated as a mixture of its isomers by simple distillation (3.7 T, 64 OC); 84.1% yield. GC/MS(EI): m/z (%) 150(22), 135(2), 122(18), 106(14), 93(100), 79(73), 65(25), 53(25), 41(44). ¹H NMR (CDCl₃): δ 9.64-9.67 (mixture of doublets for CHO). ¹³C NMR (CDCl₃): δ 203.1 and 202.7 for CHO.

### Example 2

This Example illustrates the synthesis of the mixture of ((1S,4S)-5-ethylidenebicyclo[2.2.1]heptan-2-yl)methanol and ((1S,4R)-6-ethylidenebicyclo[2.2.1]heptan-2-yl)methanol:

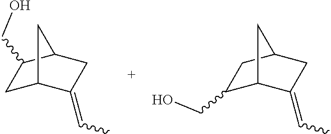

Sodium borohydride (8.3 g, 1.1 molar equivalents) was added in portions at 0° C. to the product of Example 1 (30.0 g) in 180 mL methanol. One hour after the addition of the reducing agent, the reaction was quenched while cold with water and allowed to warm to room temperature. Methanol was removed under reduced pressure and the remaining mixture was extracted twice with diethyl ether. The combined extracts were washed with 5% HCl, then with brine and concentrated. After Kugelrohr distillation (1.6 T, 106° C.), 29.5 g of product, as a mixture of its isomers, was obtained (97.0% yield). GC/MS(EI): m/z (%) 152(30), 134(6), 121(25), 105(22), 93(100), 79 (85), 67(28), 55(29), 41(69). ¹H NMR (CDCl₃): δ 5.03-5.25 (vinyl proton).

### Example 3

This Example illustrates the synthesis of the mixture of 1-((1S,4S)-5-ethylidenebicyclo[2.2.1]heptan-2yl)ethanol and 1-((1S,4R)-6-ethylidenebicyclo[2.2.1]heptan-2yl)ethanol:

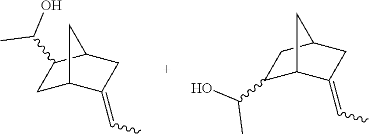

The product of Example 1 (49.9 g) was added drop-wise to a 3.0M solution of methylmagnesium bromide in diethyl ether (133 mL, 1.2 molar equivalents) at a rate so that the temperature of the reaction remained below 40° C. Twenty minutes after the addition, the reaction was quenched with cold saturated aqueous ammonium chloride. The quenched material was extracted twice with diethyl ether. The combined extracts were washed with brine, dried over MgSO₄ and concentrated to 50.0 g of oil. After simple distillation (1.8 T, 63° C.), 43.9 g of product, as a mixture of its isomers, was obtained (79.6% yield). GC/MS(EI): m/z (%) 166(30), 148(8), 133(28), 119(25), 105(24), 93(100), 79(88), 67(35), 55(29), 45(75).

### Example 4

This Example illustrates the synthesis of the mixture of 1-((1S,4S)-5-ethylidenebicyclo[2.2.1]heptan-2yl)propan-1-ol and 1-((1S,4R)-6-ethylidenebicyclo[2.2.1]heptan-2yl)propan-1-ol:

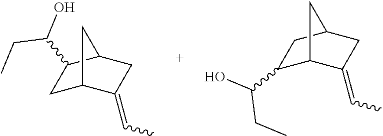

Conditions are the same as for Example 3, but with ethylmagnesium bromide (3.0M in diethyl ether) as the Grignard reagent; 80.6% yield. GC/MS(EI): m/z (%) 180(29), 162(6), 151(8), 133(40), 119(13), 105(28), 93(100), 79(81), 67(38), 59(58), 53(23), 41(70).

### Example 5

This Example illustrates the synthesis of the mixture of 1-((1S,4S)-5-ethylidenebicyclo[2.2.1]heptan-2yl)prop-2-en-1-ol and 1-((1S,4R)-6-ethylidenebicyclo[2.2.1]heptan-2yl)prop-2-en-1-ol:

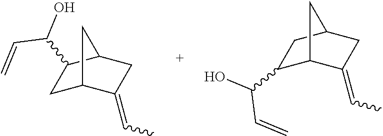

Conditions are the same as for Example 3, but with vinylmagnesium chloride (1.6M in tetrahydrofuran) as the Grignard reagent; 65.6% yield. GC/MS(EI): m/z (%) 178(3), 170(1), 162(5), 147(3), 133(4), 121(55), 115(4), 105(43), 93(100), 79(90), 65(29), 55(38), 50(5), 41(60).

### Example 6

This Example illustrates the synthesis of the mixture of 1-((1S,4S)-5-ethylidenebicyclo[2.2.1]heptan-2yl)-2-methylpropan-1-ol and 1-((1S,4R)-6-ethylidenebicyclo[2.2.1]heptan-2yl)-2-methylpropan-1-ol:

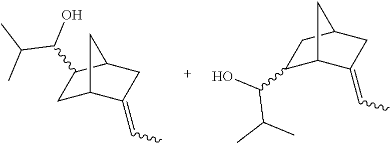

Conditions are the same as for Example 3, but with isopropylmagnesium chloride (2.0M in tetrahydrofuran) as the Grignard reagent, and column chromatography for purification (5% ethyl acetate in hexanes); 38.8% yield. GC/MS(EI): m/z (%) 194(36), 176(4), 161(4), 151(31), 133(79), 121(13), 107(48), 93(100), 79(85), 67(33), 55(63), 41(81).

### Example 7

This Example illustrates the synthesis of the mixture of 1-((1S,4S)-5-ethylidenebicyclo[2.2.1]heptan-2yl)ethyl acetate and 1-((1 S, 4R)-6-ethylidenebicyclo[2.2.1]heptan-2yl)ethyl acetate:

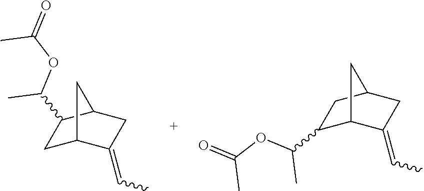

The product of Example 3 (43.9 g) was combined with acetic anhydride (35.1 g, 1.3 molar equivalents) and toluene (45 ml) and stirred at reflux for 12 hours. After cooling, the reaction was quenched with water and extracted twice with diethyl ether. The combined extracts were washed NaHCO₃ (sat.), with brine, dried over MgSO₄ and concentrated. After fractional distillation (1.8 T, 82° C.), 39.0 g of product, as a mixture of its isomers, was obtained (70.8% yield). GC/MS(EI): m/z (%) 208(6), 193(1), 148(13), 133(19), 119(25), 105(19), 93(36), 79(25), 65(6), 55(11), 43(100).

### Example 8

This Example illustrates the synthesis of the mixture of 1-((1S,4S)-5-ethylidenebicyclo[2.2.1]heptan-2yl)propyl acetate and 1-((1S,4R)-6-ethylidenebicyclo[2.2.1]heptan-2yl)propyl acetate:

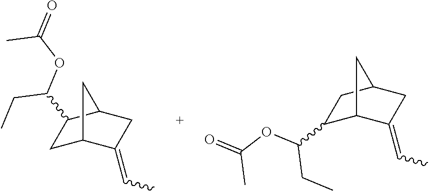

Conditions are the same as for Example 7, but with the product of Example 4 as the starting material and column chromatography for purification (2% ethyl acetate in hexanes); 91.8% yield. GC/MS(EI): m/z (%) 222(1), 207(1), 162(20), 147(6), 133(18), 121(13), 106(28), 93(48), 79(33), 65(10), 55(13), 43(100).

### Example 9

This Example illustrates the synthesis of the mixture of 1-((1S,4S)-5-ethylidenebicyclo[2.2.1]heptan-2yl)allyl acetate and 1-((1S,4R)-6-ethylidenebicyclo[2.2.1]heptan-2yl)allyl acetate:

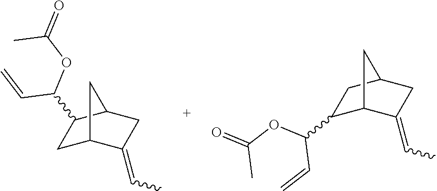

Conditions are the same as for Example 7, but with the product of Example 5 as the starting material and column chromatography for purification (2% ethyl acetate in hexanes); 93.1% yield. GC/MS(EI): m/z (%) 220(4), 207(1), 191(1), 178(4), 160(13), 145(13), 131(13), 119(25), 105(19), 93(85), 79(56), 67(13), 55(13), 43(100).

### Example 10

This Example illustrates the synthesis of the mixture of 1-((1S,4S)-5-ethylidenebicyclo[2.2.1]heptan-2yl)-2-methylpropyl acetate and 1-((1S,4R)-6-ethylidenebicyclo[2.2.1]heptan-2yl)-2-methylpropyl acetate:

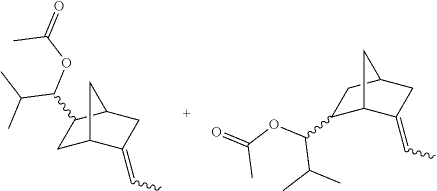

Conditions are the same as for Example 7, but with the product of Example 6 as the starting material and bulb-to-bulb distillation for purification (0.6 T, 108° C.); 95.7% yield. GC/MS(EI): m/z (%) 236(4), 221(1), 193(1), 176(7), 161(10), 147(5), 133(19), 120(10), 105(15), 93(26), 79(23), 67(7), 55(13), 43(100).

### Example 11

This Example illustrates the synthesis of the mixture of 1-((1S,4S)-5-ethylidenebicyclo[2.2.1]heptan-2yl)ethanone and 1-((1S,4R)-6-ethylidenebicyclo[2.2.1]heptan-2yl)ethanone:

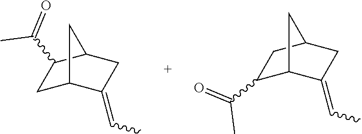

The product of Example 3 (10.0 g) dissolved in anhydrous methylene chloride (100 mL) was combined with (2,2,6,6-Tetramethylpiperidin-1-yl)oxyl (TEMPO) (0.97 g, 0.1 molar equivalents) at room temperature. (Bisacetoxy)iodobenzene (BAIB) (20.1 g, 1.1 molar equivalents) was added batch-wise, and the reaction was stirred for 18 hours. The reaction was then quenched with Na₂S₂O₃ (sat.) and the organic and aqueous layers separated. The aqueous portion was extracted twice further with methylene chloride. The combined organic extracts were washed NaHCO₃ (sat.), with brine, dried over MgSO₄ and concentrated. Column chromatography (2% ethyl acetate in hexanes) provided 5.8 g of product, as a mixture of its isomers (58.4% yield). GC/MS(EI): m/z (%) 164(18), 149(13), 135(28), 121(14), 106(13), 93(48), 79(40), 71(15), 65(11), 55(13), 43(100).

### Example 12

This Example illustrates the synthesis of the mixture of 1-((1S,4S)-5-ethylidenebicyclo[2.2.1]heptan-2yl)propan-1-one and 1-((1S,4R)-6-ethylidenebicyclo[2.2.1]heptan-2yl)propan-1-one:

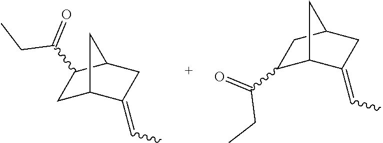

Conditions are the same as for Example 11, but with the product of Example 4 as the starting material; 82.5% yield. GC/MS(EI): m/z (%) 178(10), 163(1), 121(61), 106(13), 93(100), 85(35), 79(69), 65(15), 57(38), 41(31).

### Example 13

This Example illustrates the synthesis of the mixture of 1-((1S,4S)-5-ethylidenebicyclo[2.2.1]heptan-2yl)prop-2-en-1-one and 1-((1S,4R)-6-ethylidenebicyclo[2.2.1]heptan-2yl)prop-2-en-1-one:

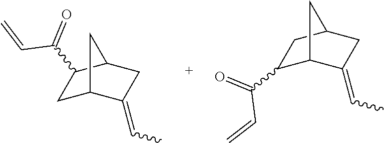

The product of Example 5 (1.5 g) dissolved in anhydrous methylene chloride (5 mL) was added drop-wise to pyridinium chlorochromate (PCC) (4.3 g, 2.5 molar equivalents) dissolved in anhydrous methylene chloride (20 mL) over 3.6 g of 3 Å mol sieves. After stirring at room temperature for 1 hour, the reaction was treated with 50 mL diethyl ether and filtered through celite. The ether washings were concentrated. Bulb-to-bulb distillation (2.5 T, 100° C.) provided 0.3 g of product, as a mixture of its isomers (20.3% yield). GC/MS(EI): m/z (%) 176(10), 161(5), 147(19), 133(5), 121(15), 106(19), 93(74), 79(60), 67(25), 55(100), 41(38).

### Example 14

This Example illustrates the synthesis of the mixture of 1-((1S,4S)-5-ethylidenebicyclo[2.2.1]heptan-2yl)-2-methylpropan-1-one and 1-((1S,4R)-6-ethylidenebicyclo[2.2.1]heptan-2yl)-2-methylpropan-1-one:

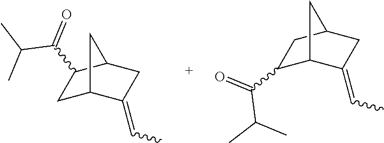

Conditions are the same as for Example 13, but with the product of Example 6 as the starting material; 52.3% yield after bulb-to-bulb distillation (1.6 T, 102° C.). GC/MS(EI): m/z (%) 192(6), 177(1), 163(4), 149(6), 133(1), 121(55), 105(8), 93(80), 79(53), 67(15), 55(19), 43(100).

### Example 15

This Example illustrates the synthesis of the mixture of 5-ethylidenebicyclo[2.2.1]heptane-2-carbaldehyde oxime and 6-ethylidenebicyclo[2.2.1]heptane-2-carbaldehyde oxime:

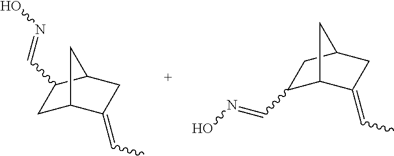

The product of Example 1 (50.0 g) was added drop-wise to a mixture of hydroxyl amine hydrochloride (39.4 g, 1.7 molar equivalents), pyridine (52.7 g, 2.0 molar equivalents) in ethanol (1 L) and heated at reflux for 20 hours. After cooling, the ethanol was removed under reduced pressure. The reaction mixture was treated with 100 mL water and extracted with 2×50 mL ethyl acetate. The combined extracts were washed with 1N HCl, then then brine, and dried over MgSO₄. After concentration and drying under vacuum, 55.3 g of crude product, as a mixture of its isomers, was obtained (quantitative yield). The crude product was used without purification for the following step. GC/MS(EI): m/z (%) 165(13), 148(19), 134(9), 120(13), 106(30), 91(69), 79(75), 67(25), 56(100), 41(44).

### Example 16

This Example illustrates the synthesis of the mixture of (1S,4S)-5-ethylidenebicyclo[2.2.1]heptane-2-carbonitrile and (1S,4R)-6-ethylidenebicyclo[2.2.1]heptane-2-carbonitrile:

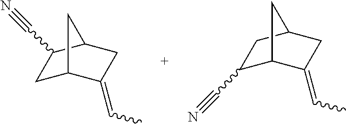

The product of Example 15 (47.0 g) was combined with potassium phosphate (4.7 g, 10% wt.) in xylenes (100 mL) and heated at reflux for 24 hours, with water collecting in a Dean-Stark trap. After cooling, the reaction was quenched with water and extracted with ethyl acetate. The combined extracts were dried over MgSO₄ and concentrated. Simple distillation (0.5 T, 100° C.) gave 37.0 g of product, as a mixture of its isomers (88.5% yield). GC/MS(EI): m/z (%) 147(19), 132(7), 118(5), 107(23), 93(80), 79(100), 65(25), 51(30), 41(33).

### Example 17

This Example illustrates the synthesis of the mixture of 3-((1S,4S)-5-ethylidenebicyclo[2.2.1]heptan-2-yl)-2-methylacrylaldehyde and 3-((1S,4S)-6-ethylidenebicyclo[2.2.1]heptan-2-yl)-2-methylacrylaldehyde:

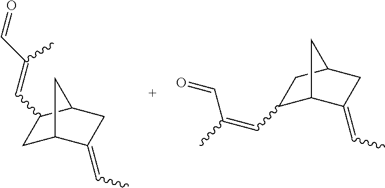

The product of Example 1 (10.0 g) was dissolved in methanol (20 mL) and NaOH (6.4 g of a 50% aqueous solution, 1.2 molar equivalents) was added. Propanal (1.0 molar equivalent) was then added drop-wise. After 30 minutes the reaction was quenched with NH₄HCl (sat.) and extracted with diethyl ether. The extracts were dried over MgSO₄ and concentrated. Column chromatography (1% ethyl acetate in hexanes) provided 5.9 g of product, as a mixture of its isomers (46.6% yield). GC/MS(EI): m/z (%) 190(3), 175(1), 161(3), 147(4), 133(4), 119(6), 106(19), 91(56), 79(90), 65(38), 53(48), 41(100).

### Example 18

This Example illustrates the synthesis of the mixture of 3-((1S,4S)-5-ethylidenebicyclo[2.2.1]heptan-2-yl)-2-methylprop-2-en-1-ol and 3-((1S,4S)-6-ethylidenebicyclo[2.2.1]heptan-2-yl)-2-methylprop-2-en-1-ol:

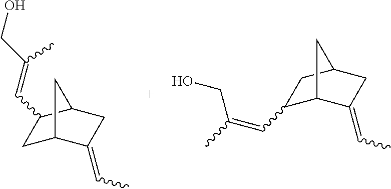

The product of Example 17 (0.15 g) was dissolved in methanol (2 mL) and cooled to 5° C. NaBH₄ (0.04 g, 1.5 molar equivalents) was added and the reaction was allowed to gradually warm to room temperature. The reaction was quenched with water and extracted with diethyl ether. The extracts were dried over MgSO₄ and concentrated. Column chromatography (10% ethyl acetate in hexanes) provided 0.1 g of product, as a mixture of its isomers (66.7% yield). GC/MS(EI): m/z (%) 192(13), 174(13), 161(40), 145(13), 133(18), 119(35), 106(63), 93(94), 79(100), 67(25), 55(28), 41(68).

### Example 19

This Example illustrates the synthesis of the mixture of (1S,4S)-2-ethylidene-5-((4Z)-hepta-1,4-dienyl)bicyclo[2.2.1]heptane and (1S,4S)-2-ethylidene-6-((4Z)-hepta-1,4-dienyl)bicyclo[2.2.1]heptane:

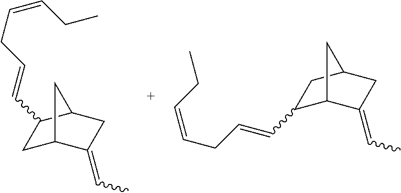

Cis-3-hexenyltriphenylphosphonium bromide (15.6 g, 1.1 molar equivalents) was added to a 500 mL flask. Anhydrous diethyl ether (92 mL) was added and stirred to make a slurry. N-Butyl lithium (1.6M in diethyl ether, 22.3 mL, 1.1 molar equivalents) was added to the slurry drop-wise at room temperature. After 2 hours, the orange solution was cooled to −15° C., and the product of Example 1 (5.0 g) with diethyl ether (30 mL) was added drop-wise. The solution was allowed to gradually warm to room temperature and stirred for 18 hours. The reaction was then quenched with NH₄HCl (sat.) and extracted with diethyl ether. The extracts were washed with brine, dried over MgSO₄ and concentrated. Bulb-to-bulb distillation provided 1.5 g of product, as a mixture of its isomers (20.8% yield). GC/MS(EI): m/z (%) 216(13), 201(4), 187(19), 173(10), 159(10), 147(31), 131(13), 119(25), 106(50), 91(58), 79(100), 67(30), 55(29), 41(56).

### Example 20

This Example illustrates the synthesis of the mixture of (1S,4S)-2-(allyloxymethyl)-5-ethylidenebicyclo[2.2.1]heptane and (1 S,4R)-2-(allyloxymethyl)-6-ethylidenebicyclo[2.2.1]heptane:

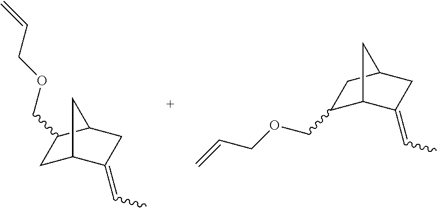

The product of Example 2 (9.8 g) was added drop-wise at room temperature to a slurry of sodium hydride (1.6 g, 1.0 molar equivalents) in anhydrous THF (100 mL). The mixture was refluxed for 3 hours, then cooled to 45° C., at which point allyl chloride (6.5 mL, 1.2 molar equivalents) was added drop-wise. After 2 hours at 45° C., the reaction was refluxed for another 18 hours. The starting alcohol was only 55% converted, but was then quenched with water and extracted with diethyl ether. The extracts were washed with brine, dried over MgSO₄ and concentrated. Column chromatography (2% ethyl acetate in hexanes) provided 4.5 g of product, as a mixture of its isomers (65.6% yield based on 55% conversion). GC/MS(EI): m/z (%) 192(6), 177(1), 163(1), 151(4), 135(8), 121(63), 112(8), 107(27), 93(90), 79(73), 67(25), 55(29), 41(100).

### Example 21

This Example illustrates the synthesis of the mixture of (1S,4S)-2-ethylidene-5-((2-methylallyloxy)methyl)bicyclo[2.2.1]heptane and (1 S,4R)-2-ethylidene-6-((2-methylallyloxy)methyl)bicyclo[2.2.1]heptane:

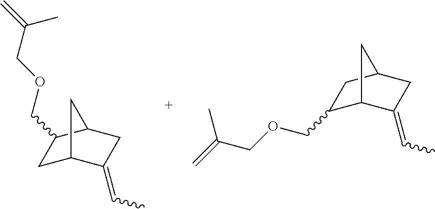

Conditions are the same as for Example 20, but with methallyl chloride as the alkyl halide added drop-wise at 45° C.; 91.1% yield based on 100%/conversion. GC/MS(EI): m/z (%) 206(19), 191(1), 177(1), 163(1), 150(6), 135(38), 119(25), 105(32), 93(100), 79(81), 67(19), 55(69), 41(45).

### Example 22

This Example illustrates the synthesis of the mixture of 4-(((1S,4S)-5-ethylidenebicyclo[2.2.1]heptan-2-yl)methoxy)butanal and 4-(((1 S,4R)-6-ethylidenebicyclo[2.2.1]heptan-2-yl)methoxy)butanal:

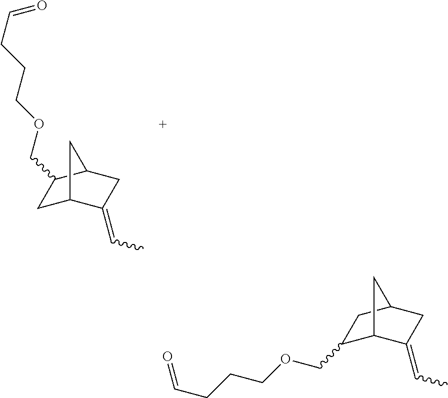

The product of Example 20 (0.5 g), Rh(CO)₂(Acac) (1.0 mg, 0.0015 molar equivalents), NIXANTPHOS (0.1 g, 0.0078 molar equivalents) and 5 mL toluene were sealed in an autoclave. The vessel was purged with a mixture of 1:1 CO:H₂ gases, then filled to 250 psi and heated to 65° C. After 18 hours, the product (0.37 g) was isolated as a mixture of its isomers by simple distillation (1.1 T, 110° C.); 64.9% yield. GC/MS(EI): m/z (%) 222(6), 207(1), 194(1), 165(4), 152(23), 134(13), 121(56), 106(35), 93(100), 79(88), 67(25), 55(25), 41(88).

### Example 23

This Example illustrates the synthesis of the mixture of 4-(((1S,4S)-5-ethylidenebicyclo[2.2.1]heptan-2-yl)methoxy)butan-1-ol and 4-(((1S,4R)-6-ethylidenebicyclo[2.2.1]heptan-2-yl)methoxy)butan-1-ol:

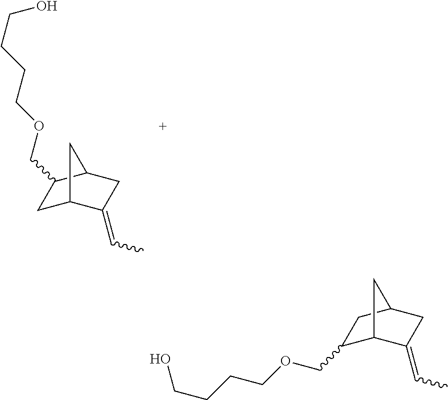

Sodium borohydride (0.08 g, 1.1 molar equivalents) was added at 0° C. to the product of Example 22 (0.5 g) in 2 mL methanol. One hour after the addition of the reducing agent, the reaction was quenched while cold with water and allowed to warm to room temperature. Methanol was removed under reduced pressure and the remaining mixture was extracted twice with diethyl ether. The combined extracts were concentrated and Kugelrohr distillation gave 0.1 g of product, as a mixture of its isomers (33.3% yield).

### Example 24

This Example illustrates the synthesis of the mixture of (1S,4S)-2-((1-ethoxyethoxy)methyl)-5-ethylidenebicyclo[2.2.1]heptane and (1S,4R)-2-((1-ethoxyethoxy)methyl)-6-ethylidenebicyclo[2.2.1]heptane:

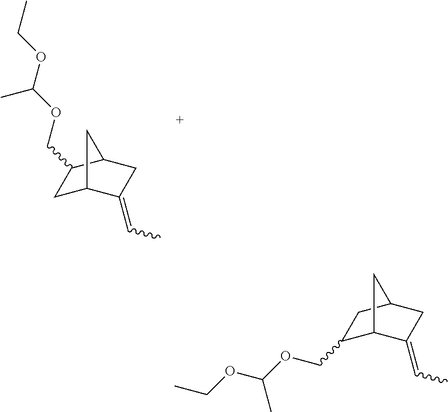

The product of Example 2 (0.5 g) was combined at room temperature with ethyl vinyl ether (0.35 g, 1.5 molar equivalents) neat. Amberlyst 15 (6.0 mg) was added and the reaction turned yellow. After 1 hour the reaction was filtered and distilled (bulb-to-bulb) to give 0.23 g of a mixture of the desired products and the vinyl ether. Column chromatography (1-5% ethyl acetate in hexanes) provided 0.01 g of product, as a mixture of its isomers (1.5% yield for evaluation). GC/MS(EI): m/z (%) 224(4), 209(1), 195(1), 178(13), 163(11), 151(6), 135(23), 119 (13), 105(19), 93(56), 79(63), 73(100), 67(19), 53(19), 45(95).

### Example 25

This Example illustrates the synthesis of the mixture of ((1S,4S)-5-ethylidenebicyclo[2.2.1]heptan-2-yl)methyl isobutyrate and ((1 S,4R)-6-ethylidenebicyclo[2.2.1]heptan-2-yl)methyl isobutyrate:

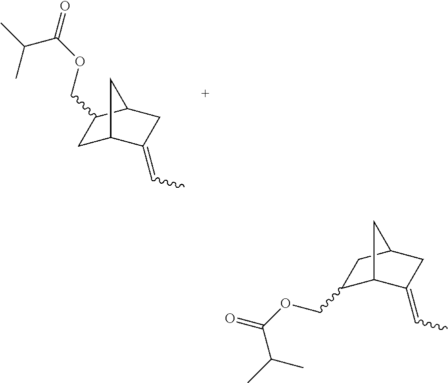

The product of Example 2 (1.0 g) was combined with 4-dimethylaminopyridine (0.08 g, 0.1 molar equivalents) and 10 mL methylene chloride at room temperature. After cooling the reaction to 5° C., pyridine (1.0 g, 2.0 molar equivalents) was added. Isobutyryl chloride (1.05 g, 1.5 molar equivalents) was then added drop-wise. The reaction was allowed to warm to room temperature and stirred for 18 hours. The reaction was then quenched with water and extracted with methylene chloride. The combined extracts were washed with 5% HCl, water and then NaHCO₃ (sat.). After drying over MgSO₄ and concentrating, 1.2 g of crude product was obtained. Kugelrohr distillation (1.0 T, 120° C.) provided 0.8 g of product, as a mixture of its isomers (54.8% yield). GC/MS(EI): m/z (%) 222(6), 207(1), 193(1), 152(1), 134(38), 119(65), 105(44), 93(73), 79(63), 71(31), 55(15), 43(100).

### Example 26

This Example illustrates the synthesis of the mixture of ((1S,4S)-5-ethylidenebicyclo[2.2.1]heptan-2-yl)methyl methyl carbonate and ((1S,4R)-6-ethylidenebicyclo[2.2.1]heptan-2-yl)methyl methyl carbonate:

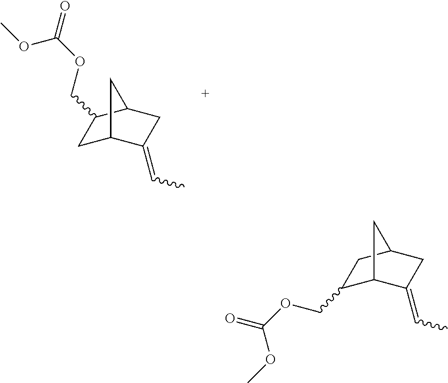

The product of Example 2 (1.0 g) was combined 5 mL THF at room temperature. After cooling the reaction to 5° C., methyl chloroformate (0.69 g, 1.12 molar equivalents) was added, followed by pyridine (0.58 g, 1.12 molar equivalents) drop-wise. The reaction was allowed to warm to room temperature and stirred for 18 hours. The reaction was then quenched with water and extracted with methylene chloride. The combined extracts were washed with 5% HCl, water and then NaHCO₃ (sat.). After drying over MgSO₄ and concentrating, 1.7 g of crude product was obtained. Kugelrohr distillation (1.5 T, 118° C.) provided 1.0 g of product, as a mixture of its isomers (72.5% yield). GC/MS(EI): m/z (%) 210(13), 195(1), 153(1), 134(44), 119(54), 106(63), 91(94), 79(100), 59(35), 41(55).

### Example 27

This Example illustrates the synthesis of the mixture of ethyl ((1 S,4S)-5-ethylidenebicyclo[2.2.1]heptan-2-yl)methyl carbonate and ethyl ((1S,4R)-6-ethylidenebicyclo[2.2.1]heptan-2-yl)methyl carbonate:

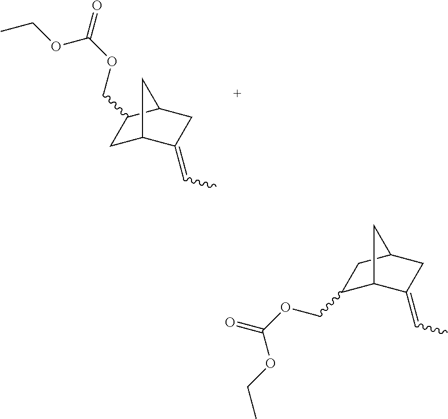

Conditions are the same as for Example 26, but with ethyl chloroformate; 65.3% yield. GC/MS(EI): m/z (%) 224(6), 195(1), 152(1), 134(56), 119(56), 106(63), 93(100), 79(98), 67(19), 55(19), 41(48).

### Example 28

A perfume composition with a berry rhubarb type note was prepared from the compounds of EXAMPLE 7 to demonstrate their use in a candle. The composition of the perfume is summarized in Table 6.

TABLE 6

| | PARTS PER |
|---|---|
| COMPONENT | THOUSAND |
| Anisyl acetone | 25 |
| Benzyl acetate | 12 |
| Benzyl benzoate | 503 |
| BHT | 10 |
| Citronellyl propionate, L. | 4 |
| Damascone, Delta | 7.5 |
| Dimethyl sulfide @ 1% in BB | 3.5 |
| Dioctyl adipate | 250 |
| Ethyl-2-methylbutyrate | 3 |
| Ethyl acetate | 3.5 |
| Ethyl fraison | 4 |
| Ethyl methyl phenylglycidate | 4 |
| Fructalate ™ | 18 |
| Hexen-1-ol, cis-3 | 4 |
| Hexenyl acetate, cis-3 | 3 |
| Ionone, Beta | 9 |
| Methyl dioxolan (Fructone) | 16 |
| Methyl octine carbonate | .5 |
| EXAMPLE 7 -- 1-((1S,4S)-5- | 50 |
| ethylidenebicyclo[2.2.1]heptan-2yl)ethyl acetate and 1- | |
| ((1S,4R)-6-ethylidenebicyclo[2.2.1]heptan-2yl)ethyl | |
| acetate | |
| Prenyl acetate | 4 |
| Raspberry ketone | 56 |
| Ringonol ™ 50 TEC @ .1% in BB | 1 |
| Veltol Plus | 9 |
| Total | 1000 |

This composition using the compounds of EXAMPLE 7 showed superior character and equal or better strength over compositions using known materials to achieve a similar berry/rhubarb type odor. When the compounds of EXAMPLE 7 were substituted with styrallyl acetate, the fragrance was less fresh and natural. When the compounds of EXAMPLE 7 were replaced with Rhubafuran, the character was not as pleasant, but the strength was similar. When the compounds of EXAMPLE 7 were substituted with Gardamide, the fragrance was weaker.

### Example 29

A perfume composition with a shiso/perilla type note was prepared from the compounds of EXAMPLE 16 to demonstrate their use in a liquid electric air freshener. The composition of the perfume is summarized in Table 7.

TABLE 7

| | PARTS PER |
|---|---|
| COMPONENT | THOUSAND |
| Aldehyde C-10 (decanal) | 3 |
| Aldehyde C-12 MNA | 1.5 |
| Aldehyce C-8 (octanal) | 1 |
| Allyl heptoate | 1.5 |
| BHT | 5 |
| Borneol, L. crystal | 1 |
| Bornyl acetate, L. | .5 |
| Citral synthetic refined | 15 |
| Clove leaf oil, natural EO | 9 |
| Decenal, trans-4 | .5 |
| Dihydromyrcenol | 8 |
| Dimethyl anthranilate | 2 |
| Dodecenl, trans-2 | 2.5 |
| Dowanol ™ DPMA | 653.5 |
| Ethyl-2-methylbutyrate | 1 |
| Ethyl butyrate | .5 |
| Ethyl linalool | 70 |
| Eucalyptol | 1 |
| Floropal ® LCV (Florexan) | 20 |
| Geraniol intermediate 60 | 32 |
| Geranyl acetate extra | 4 |
| Ginger topnote CO₂ natural extract | 5 |
| Grapefruit oil expressed natural EO | 20 |
| Hedione | 45 |
| Hexen-1-ol, cis-3 | 2 |
| Limonene thiol 1% TEC @ 10% TEC | 2.5 |
| Linalyl acetate synthetic | 20 |
| Melonal | .5 |
| Methylpamplemousse | 6 |
| EXAMPLE 16 -- (1S,4S)-5- | 50 |
| ethylidenebicyclo[2.2.1]heptane-2-carbonitrile oxime | |
| and (1S,4R)-6-ethylidenebicyclo[2.2.1]heptane-2- | |
| carbonitrile | |
| Orange oil 10-fold c'less | 7.5 |
| Pettigrain Oil Paraguay Natural EO | 2 |
| Pharaone ® 10% DPG | 1 |
| Rhubafuran ® | .5 |
| Terpineol 900 alpha | 3 |
| Terpinyl acetate | 1.5 |
| Thymol crystals | 1 |
| Total | 1000 |

This composition was tested against the base composition alone, i.e., the perfume composition of Table 7 without the compound of Example 16, as well as the perfume composition of Table 7 comprising perilla aldehyde instead of the compound of Example 16 at an amount of about 0.3% by weight. The base composition alone gave a yuzu-ginger fragrance. The perfume with perilla aldehyde gave spicy, fresh, cumin-like notes.

This composition using the compounds of EXAMPLE 16 (at an amount of about 5% by weight, an amount comparable to the price of 0.3% perilla aldehyde) showed similar strength and character to perilla aldehyde, but at significantly less cost. In addition, the compounds of EXAMPLE 16 have shown excellent stability in a variety of base types (see EXAMPLE 30).

### Example 30

The compounds of EXAMPLE 16 were formulated into base types including fabric softener, hair color, shampoo, body soap and alcohol solution. Each sample was tested for four weeks at various temperature. The samples were evaluated for odor, appearance and strength.

### A. Materials and Methods

Evaluations were performed by 5 Perfumers/Evaluators. Each base was dosed with the amount shown of the compounds of Example 16. Each sample was stored at the three temperatures and compared with the control base alone at 5° C. after 4 weeks. Formulations for each base are summarized in Tables 8-11.

TABLE 8
Fabric Softener Formulation

| FABRIC SOFTENER FORMULATION | (g) |
|---|---|
| LION SOFTER EQ | 15.00 |
| LEOCOL TDA-400-75 | 2.00 |
| CALSIUM CHLORIDE | 0.05 |
| DISODIUM LAURETH SULFOSUCCINATE | 10.00 |
| BHT | 0.01 |
| SODIUM HYDROXIDE | 0.01 |
| ALCOHOL | 3.00 |
| SAMPLE | 0.40 |
| WATER | balance |
| TOTAL | 100.00 |

TABLE 9
Hair Color Formulation

| HAIR COLOR FORMULATION | (g) |
|---|---|
| CETETH-20 | 3.00 |
| CETYL ALCOHOL | 7.00 |
| MINERAL OIL | 2.00 |
| STEARIC ACID | 3.00 |
| SODIUM SULFITE | 0.10 |
| EDTA-2Na | 0.10 |
| AMMONIUM HYDROXIDE | 8.00 |
| SAMPLE | 0.10 |
| WATER | balance |
| TOTAL | 100.00 |

TABLE 10
Shampoo Formulation

| SHAMPOO FORMULATION | (g) |
|---|---|
| SODIUM LAURETH SULFATE | 14.00 |
| LAURAMIDOPROPYL BETAINE | 4.00 |
| COCAMIDE DEA | 3.00 |
| CATIONIC CELLULOSE | 0.50 |
| GLYCOL DISTEARATE | 1.00 |
| ETHYL PARABEN | 0.25 |
| CITRIC ACID | appropriate |
| SAMPLE | 0.50 |
| WATER | balance |
| TOTAL | 100.00 |

TABLE 11
Body Soap Formulation

| BODY SOAP FORMULATION | (g) |
|---|---|
| TRIETHANOLAMINE | 9.00 |
| LAURIC ACID | 6.00 |
| MYRISTIC ACID | 9.00 |
| DISODIUM LAURETH SULFOSUCCINATE (1E.O) | 10.00 |
| DECYL GLUCOSIDE | 8.00 |
| GLYCERYL LAURATE | 1.00 |
| GLYCOL DISTEARATE | 2.50 |
| COCAMIDE DEA | 3.00 |
| PG | 5.00 |
| BHT | 0.05 |
| EDTA-2Na | 0.10 |
| ETHYL PARABEN | 0.20 |
| METHYL PARABEN | 0.10 |
| SAMPLE | 1.00 |
| WATER | balance |
| TOTAL | 100.00 |

### B. Results

For evaluation criteria, scores of D and E or 4 and 5 are problematic. Scores of C or 3 are at tolerance level, and scores of A and B or 1 and 2 are acceptable with no problem.

Odor after 4 Weeks

### Evaluation Criteria for Odor:

A: No Change

B: Very Slight Change

C: Slight Change

D: Change

E: Significant Change

The results of the odor evaluation are summarized in Table 12.

TABLE 12

| | Base | | | | | | | | | | | | | | |
|---|---|---|---|---|---|---|---|---|---|---|---|---|---|---|---|
| | | | | | | | | | | | | | Alcohol | | |
| | Fabric softener | | | Hair color | | | Shampoo | | | Body soap | | | solution | | |
| | Dosage | | | | | | | | | | | | | | |
| | 0.4% | | | 0.4% | | | 0.5% | | | 1.0% | | | 10.0% | | |
| Sample | 5° C. | RT | 45° C. | 5° C. | RT | 45° C. | 5° C. | RT | 45° C. | 5° C. | RT | 45° C. | 5° C. | RT | 45° C. |
| EX. 16 | A | A | B | A | A | C | A | A | C | A | A | B | A | A | C |

Appearance after 4 Weeks

### Evaluation Criteria for Color:

1: No Change

2: Very Slight Change

3: Slight Change

4: Change

5: Significant Change

The results of the appearance evaluation are summarized in Table 13.

TABLE 13

| | Base | | | | | | | | | | | | | | |
|---|---|---|---|---|---|---|---|---|---|---|---|---|---|---|---|
| | | | | | | | | | | | | | Alcohol | | |
| | Fabric softener | | | Hair color | | | Shampoo | | | Body soap | | | solution | | |
| | Dosage | | | | | | | | | | | | | | |
| | 0.4% | | | 0.4% | | | 0.5% | | | 1.0% | | | 10.0% | | |
| Sample | 5° C. | RT | 45° C. | 5° C. | RT | 45° C. | 5° C. | RT | 45° C. | 5° C. | RT | 45° C. | 5° C. | RT | 45° C. |
| EX. 16 | 1 | 1 | 2 | 1 | 1 | 1 | 1 | 1 | 2 | 1 | 1 | 1 | 1 | 1 | 2 |

Strength after 4 Weeks

### Evaluation Criteria for Strength:

1: No Change

2: Very Slight Change

3: Slight Change

4: Change

5: Significant Change

The results of the strength evaluation are summarized in Table 14.

TABLE 14

| | Base | | | | | | | | | | | | | | |
|---|---|---|---|---|---|---|---|---|---|---|---|---|---|---|---|
| | | | | | | | | | | | | | Alcohol | | |
| | Fabric softener | | | Hair color | | | Shampoo | | | Body soap | | | solution | | |
| | Dosage | | | | | | | | | | | | | | |
| | 0.4% | | | 0.4% | | | 0.5% | | | 1.0% | | | 10.0% | | |
| Sample | 5° C. | RT | 45° C. | 5° C. | RT | 45° C. | 5° C. | RT | 45° C. | 5° C. | RT | 45° C. | 5° C. | RT | 45° C. |
| EX. 16 | 1 | 1 | 1 | 1 | 1 | 1 | 1 | 1 | 1 | 1 | 1 | 1 | 1 | 1 | 1 |

The compounds of EXAMPLE 16 showed excellent stability in a variety of base types.

In addition to the various embodiments depicted and claimed, the disclosed subject matter is also directed to other embodiments having any other possible combination of the features disclosed and claimed herein. As such, the particular features presented herein can be combined with each other in other manners within the scope of the disclosed subject matter such that the disclosed subject matter includes any suitable combination of the features disclosed herein. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the compounds and compositions of the disclosed subject matter without departing from the spirit or scope of the disclosed subject matter.

Thus, it is intended that the disclosed subject matter include modifications and variations that are within the scope of the appended claims and their equivalents.

1. A mixture of compounds of Formula I and Formula II: 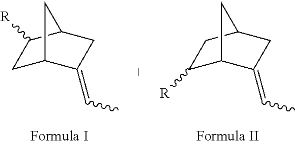
wherein R is selected from the group consisting of CH(CH₃)(OH), CH(CH₂CH₃)(OH), CH(CHCH₂)(OH), CH(CH(CH₃)₂)(OH), CH(CH₃)(OC(O)CH₃), CH(CH₂CH₃)(OC(O)CH₃), CH(CHCH₂)(OC(O)CH₃), CH(CH(CH₃)₂)(OC(O)CH₃), C(O)CH₃, C(O)CH₂CH₃, C(O)CHCH₂, C(O)(CH(CH₃)₂), CHNOH, CHC((CH₃)(CHO)), CHC((CH₃)(CH₂OH)), CHCHCH₂CHCHCH₂CH, CH₂OCH₂CHCH₂, CH₂OCH₂C((CH₂)(CH₃)), CH₂OCH₂CH₂CH₂CHO, CH₂OCH₂CH₂CH₂CH₂OH, CH₂OCH((CH₃)(OCH₂CH₃)), CH₂OCOCH(CH₃)₂, CH₂OCOOCH₃, and CH₂OCOOCH₂CH₃, and stereoisomers thereof.

2. The mixture of claim 1, wherein R is CH(CH₃)(OH), CH(CH₂CH₃)(OH), CH(CH₃)(OC(O)CH₃), or CH₂OCH₂CH₂CH₂CHO.

3. A fragrance composition comprising:
a) a mixture of compounds of Formula I and Formula II: 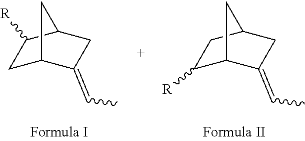
wherein R is selected from the group consisting of CH(CH₃)(OH), CH(CH₂CH₃)(OH), CH(CHCH₂)(OH), CH(CH(CH₃)₂)(OH), CH(CH₃)(OC(O)CH₃), CH(CH₂CH₃)(OC(O)CH₃), CH(CHCH₂)(OC(O)CH₃), CH(CH(CH₃)₂)(OC(O)CH₃), CN, C(O)CH₃, C(O)CH₂CH₃, C(O)CHCH₂, C(O)(CH(CH₃)₂), CHNOH, CHC((CH₃)(CHO)), CHC((CH₃)(CH₂OH)), CHCHCH₂CHCHCH₂CH, CH₂OCH₂CHCH₂, CH₂OCH₂C((CH₂)(CH₃)), CH₂OCH₂CH₂CH₂CHO, CH₂OCH₂CH₂CH₂CH₂OH, CH₂OCH((CH₃)(OCH₂CH₃)), CH₂OCOCH(CH₃)₂, CH₂OCOOCH₃, and CH₂OCOOCH₂CH₃, and stereoisomers thereof; and
b) at least one perfume ingredient selected from the group consisting of one or more bases, solvents and combinations thereof.

4. The fragrance composition of claim 3, wherein the mixture of compounds of Formula I and Formula II are present in the fragrance composition at an amount of from about 0.01% to about 50% w/w.

5. The fragrance composition of claim 4, wherein the mixture of compounds of Formula I and Formula II are present in the fragrance composition at an amount of from about 0.1% to about 10% w/w.

6. The fragrance composition of claim 5, wherein the mixture of compounds of Formula I and Formula II are present in the fragrance composition at an amount of about 5% w/w.

7. The fragrance composition of claim 5, wherein the mixture of compounds of Formula I and Formula II are present in the fragrance composition at an amount of about 2% w/w.

8. The fragrance composition of claim 3, wherein R is CN.

9. The fragrance composition of claim 3, wherein the at least one perfume ingredient is solvent.

10. The fragrance composition of claim 9, wherein the solvent is a natural solvent.

11. The fragrance composition of claim 10, wherein the natural solvent is selected from the group consisting of vegetable oils, sunflower oil, almond oil, olive oil, avocado oil, soybean oil, safflower oil, mineral oil, orange oil, and combinations thereof.

12. A consumer product comprising the fragrance composition of claim 3.

13. The consumer product of claim 12, wherein the product is selected from the group consisting of cologne, perfume, body sprays, lotions, creams, body washes, hand soaps, shampoos, conditioners, soaps, deodorants, antiperspirants, cosmetics, shampoo, conditioner, and bath agents.

14. A method to enhance or modify the organoleptic properties of a consumer product, which method comprises contacting the consumer product with a fragrance composition as defined in claim 3.

15. A flavor composition comprising:
a) a mixture of compounds of Formula I and Formula II: 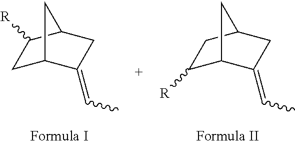
wherein R is selected from the group consisting of CH(CH₃)(OH), CH(CH₂CH₃)(OH), CH(CHCH₂)(OH), CH(CH(CH₃)₂)(OH), CH(CH₃)(OC(O)CH₃), CH(CH₂CH₃)(OC(O)CH₃), CH(CHCH₂)(OC(O)CH₃), CH(CH(CH₃)₂)(OC(O)CH₃), CN, C(O)CH₃, C(O)CH₂CH₃, C(O)CHCH₂, C(O)(CH(CH₃)₂), CHNOH, CHC((CH₃)(CHO)), CHC((CH₃)(CH₂OH)), CHCHCH₂CHCHCH₂CH, CH₂OCH₂CHCH₂, CH₂OCH₂C((CH₂)(CH₃)), CH₂OCH₂CH₂CH₂CHO, CH₂OCH₂CH₂CH₂CH₂OH, CH₂OCH((CH₃)(OCH₂CH₃)), CH₂OCOCH(CH₃)₂, CH₂OCOOCH₃, and CH₂OCOOCH₂CH₃, and stereoisomers thereof; and
b) one or more flavor co-ingredients.

16. A method to impart, enhance or modify the odor properties of a perfuming composition or perfumed consumer product, which method comprises adding to said composition or product a mixture of compounds of Formula I and Formula II: 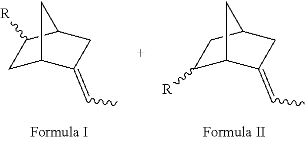
wherein R is selected from the group consisting of CH(CH₃)(OH), CH(CH₂CH₃)(OH), CH(CHCH₂)(OH), CH(CH(CH₃)₂)(OH), CH(CH₃)(OC(O)CH₃), CH(CH₂CH₃)(OC(O)CH₃), CH(CHCH₂)(OC(O)CH₃), CH(CH(CH₃)₂)(OC(O)CH₃), CN, C(O)CH₃, C(O)CH₂CH₃, C(O)CHCH₂, C(O)(CH(CH₃)₂), CHNOH, CHC((CH₃)(CHO)), CHC((CH₃)(CH₂OH)), CHCHCH₂CHCHCH₂CH, CH₂OCH₂CHCH₂, CH₂OCH₂C((CH₂)(CH₃)), CH₂OCH₂CH₂CH₂CHO, CH₂OCH₂CH₂CH₂CH₂OH, CH₂OCH((CH₃)(OCH₂CH₃)), CH₂OCOCH(CH₃)₂, CH₂OCOOCH₃, and CH₂OCOOCH₂CH₃, and stereoisomers thereof; and wherein the mixture of compounds imparts odor notes of a perilla type.

17. The method of claim 16, wherein the mixture of compounds of Formula I and Formula II are present in the composition or product at an amount of from about 0.01% to about 50% w/w.

18. The method of claim 17, wherein the mixture of compounds of Formula I and Formula II are present in the composition or product at an amount of from about 0.1% to about 10% w/w.

19. The method of claim 18, wherein the mixture of compounds of Formula I and Formula II are present in the composition or product at an amount of about 5% w/w.

20. The method of claim 19, wherein the mixture of compounds of Formula I and Formula II are present in the composition or product at an amount of about 2% w/w.

21. The method of claim 16, wherein R is CN.
